(19) United States
(12) Patent Application Publication
(10) Pub. No.: US 2026/0158255 A1
(43) Pub. Date: Jun. 11, 2026

(54) VOLUMETRIC FORMATION OF STRUCTURES

(71) Applicant: University of Central Florida Research Foundation, Inc., Orlando, FL (US)

(72) Inventors: Alexander Cockerham, Orlando, FL (US); Stephen M. Kuebler, Orlando, FL (US); Xiaoming Yu, Orlando, FL (US); He Cheng, Orlando, FL (US)

(73) Assignee: University of Central Florida Research Foundation, Inc., Orlando, FL (US)

(21) Appl. No.: 19/382,924

(22) Filed: Nov. 7, 2025

Related U.S. Application Data
(60) Provisional application No. 63/729,628, filed on Dec. 9, 2024.

(51) Int. Cl. A61M 37/00; B81C 1/00

(52) U.S. Cl. CPC A61M 37/0015; B81C 1/00111; A61M 2037/0046; A61M 2037/0053; A61M 2205/0244; A61M 2207/10; B81C 2201/0197

(57) ABSTRACT

A method includes illuminating a medium with optical illumination. The medium is optically curable to form a cured structure of a portion of the medium that is illuminated by a concentration of the optical illumination. The optical illumination is incident on the medium along an optical axis. Illuminating includes forming illumination that is both axially and transversely structured to form a volumetric structure in the medium that is contoured relative to the optical axis.

### CROSS-REFERENCE TO RELATED APPLICATIONS

This application claims benefit of priority to U.S. Provisional Patent Application No. 63/729,628 filed Dec. 9, 2024 the content of which is incorporated by reference herein in its entirety.

### GOVERNMENT RIGHTS STATEMENT

This invention was made with Government support under Contract No. 1711356, 1846671 awarded by the National Science Foundation. The Government has certain rights in this invention.

### BACKGROUND

### 1. Field

The present disclosure relates to optics, and more particularly to optical beam fabrication of three-dimensional structures.

### 2. Description of Related Art

A variety of methods are known in the traditional techniques for fabricating three-dimensional structures such as microstructures in curable or developable fabrication media. The applications include forming structures such as microneedles or microneedle arrays, and more generally forming structures additively by stacking layers of fabricated structures.

The conventional techniques have been considered satisfactory for their intended purpose. However, there is an ever-present need for improved systems and methods for optical formation of three-dimensional structures. This disclosure provides a solution for this need.

### SUMMARY

A method includes illuminating a medium with optical illumination. The medium is optically curable to form a cured structure of a portion of the medium that is illuminated by a concentration of the optical illumination. The optical illumination is incident on the medium along an optical axis. Illuminating includes forming illumination that is both axially and transversely structured to form a volumetric structure in the medium that is contoured relative to the optical axis.

The optical illumination can originate from an optical source. The volumetric structure can diverge or converge relative to the optical axis in a direction extending away from the illumination source. It is also contemplated that the volumetric structure can be convex or concave relative to the optical axis, e.g. a conical frustrum. A modulating device pattern that results in a diverging shape relative to the optical axis can made of multiple concentric anulus segments. Frustra can be formed by using modulating device pattern that are annular sections of those used to form an nth-order Bessel beam. The order of the Bessel beam can determine the width of the frustrum nearest to the source. The average radius of the annulus can determine where along the optic axis the frustrum begins. The width of the annulus can determine how rapidly the frustum “diverges.” The narrower the annulus, the more rapidly the frustrum can diverge, and the shorter it can be when measured along the optic axis. A modulating device pattern that results in a converging shape relative to the optical axis can be made of the pattern described below superposed with the phase of a converging lens.

The volumetric structure can be defined about a hollow void that extends along the optical axis. The volumetric structure can be a micro structure such as a microneedle (MN). A microneedle can be formed by a plurality of volumetric structures that are formed upon one another.

The volumetric structure can be a first volumetric structure, and the method can include illuminating the medium to form a plurality of volumetric structures. The plurality of volumetric structures can include a grid of microneedles, each formed by a single respective firing of an illuminator to illuminate a different portion of the medium. Forming each microneedle in the grid of microneedles can include positioning at least one of the medium or an optical element for each respective firing of the illuminator.

Illuminating the medium can include transmitting the illumination from an illumination source, through a modulating device such as a spatial light modulator (SLM) and diffractive optical element (DOE), and from the modulating device into the medium. The modulating device can display a phase or amplitude mask to selectively block and transmit portions of the illumination from the illuminator to the medium. The phase or amplitude mask can be predetermined to form the volumetric structure. The phase mask can include a circumferentially distributed set of spiraling curve segments. The segments can be projected to the medium at different axial locations, determined by a geometrical relationship between the radial position of the segment and the axial position on the optical axis. Each segment can be designed to generate a different beam contour at a corresponding position along the optical axis, determined by a geometrical relationship between the radial position of the segment and the axial position along the optical axis.

The volumetric structure can be the first volumetric structure in a sequence of volumetric structures. The method can include repeating illumination for each of at least one volumetric structure in the sequence of volumetric structures to stack each of the at least one volumetric structure upon a previous one of the volumetric structures in the sequence of volumetric structures starting from the first volumetric structure to form a stacked structure. Each volumetric structure in the plurality of volumetric structures in the stacked structure can have a respective predetermined size, shape, and/or contour relative to the optical axis based on a respective phase mask used for each repeat of illumination, wherein each respective predetermined size, shape, and/or contour is formed by a potentially different respective phase mask pattern on the modulating device.

A transmittance function for an annulus section of the phase mask can be

$$t ( r , φ ) = {e}^{- ik ⊥ r} {e}^{il φ} ,$$

where (r, φ) is the set of polar coordinates on the phase mask, k_{⊥} is a transverse component of the wave vector k_{⊥}=k sin α, where k=2π/λ is a wavenumber at vacuum wavelength λ, where α is angle of rays converging on the optical axis determined by an axicon base angle β and refractive index n, α=sin⁻¹(n sin β)−β, and l=0, 1, 2 . . . is an order of a Bessel mode, so that the phase mask will generate an intensity distribution in a focal region of the medium that is conical or frustoconical. Controlling the modulating device can include using feedback from the camera described below to control the illumination source and/or the modulating device.

A system includes an illuminator configured to emit a beam of illumination with a peak wavelength in a bandwidth. A modulating device such as an SLM is positioned down an optical path from the illuminator for receiving the beam of illumination from the illuminator and is configured to alter the beam of illumination into altered illumination. A layer of fabrication medium is positioned down the optical path from the modulating device. The optical path extends through the layer of fabrication medium along an optical axis. A controller is configured to perform any of the methods disclosed herein.

The system can include a telescope. The telescope can include a lens down the optical path from the modulating device and a microscope objective down the optical path from the lens. The fabrication medium can be down the optical path from the microscope objective. The lens and microscope objective can be configured to reduce size of the beam of illumination and increase peak intensity of the beam of illumination.

A β phase barium borate (BBO) crystal can be included down the optical path from the illuminator for second harmonic generation. A polarizing beam splitter (PBS) can be included down the optical path from the BBO crystal for removing fundamental light. A beam expander (BE) can be included down the optical path from the PBS, wherein the modulating device is down the optical path from the BE for expanding the beam of illumination to a diameter to overfill an aperture of the modulating device.

A beam splitter can be included between the BE and the modulating device, configured to pass the beam of illumination from the BE through the beam splitter to the modulating device, and to reflect a return of the beam of illumination from the modulating device toward the lens. A mirror down the optical path from the beam splitter can reflect the beam of illumination from the beam splitter to the lens. An iris diaphragm (ID) down the optical path from the lens can block higher-order beams diffracted from the modulating device, wherein the microscope objective is down the optical path from the ID.

A monitor microscope can include a monitoring microscope objective lens down the optical path from the layer of fabrication medium. A filter down the optical path from the monitoring microscope objective lens can block illimitation in the bandwidth of the illuminator. A camera can be included down the optical path from the filter. A camera illuminator can be included for directing camera illumination into the optical path through the beam splitter, through the telescope, through the layer of fabrication medium, through the monitor microscope objective lens, and through the filter into the camera for imaging structures in the fabrication medium.

These and other features of the systems and methods of the subject disclosure will become more readily apparent to those skilled in the art from the following detailed description of the preferred embodiments taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, preferred embodiments thereof will be described in detail herein below with reference to certain figures, wherein:

FIG. 1(a) is a schematic perspective view of an embodiment of a system constructed in accordance with the present disclosure, showing the illuminator laser and the modulating device (a spatial light modulator, SLM);

FIG. 1(b) is a schematic view showing one of four annular phase mask patterns of the modulating device (an SLM) of FIG. 1(a);

FIG. 1(c) is a schematic view showing four annular phase mask patterns, one of which is shown in FIG. 1(b), and showing a stack of cone sections in a stack formed using the four phase mask patterns;

FIG. 1(d) is a photograph of an array of cones structures formed using the system of FIG. 1(a);

FIG. 1(e) is a side-by-side comparison of an image of a simulated structure and a corresponding structure formed using the system of FIG. 1(a);

FIG. 1(f) is a photograph of a structure formed using the system of FIG. 1(a), showing a size scale for reference;

FIG. 2 is a flow chart showing sample preparations and development procedures for the system of FIG. 1(a);

FIG. 3 is a simulated x-z intensity map in the focal region for the system of FIG. 1(a), showing the five segments used to construct a cone-shaped beam;

FIG. 4 is a graph showing two orders of the Bessel modes (l) for tuning the needle beam half angle θ with a phase mask ring width w, with insets showing the phase mask and three examples of beam shape (xz-section);

FIG. 5 is a three-dimensional iso-intensity contour of the needle beam (top-most) and its five constituent segments;

FIGS. 6(a) and 6(b) are schematic perspective views of two simulated needle beams with different cone angles;

FIG. 7(a)-7(c) show a comparison of the simulated needle beam total intensity in FIG. 7(a), the dose distribution in FIG. 7(b), and a fabricated microneedle in FIG. 7(c); and

FIG. 8(a)-8(d) are scanning electron microscope (SEM) images of a microneedle array in FIG. 8(a), for individual micro needles in FIGS. 8(b) and 8(c), and for a microneedle fabricated with lower exposure dose and broken during development showing that the internal is solid in FIG. 8(d).

TABLE 1 is a spin-coater recipe chart, showing recipes used for each spin-coating procedure. Procedures 1(a) and 1(b) both have an initial step at a lower spin speed followed by a second step at a higher spin speed and longer holding time. Procedure 1(c) only has one step, so spin speed, acceleration, and hold time for step 2 are not applicable.

### DETAILED DESCRIPTION OF THE PREFERRED EMBODIMENTS

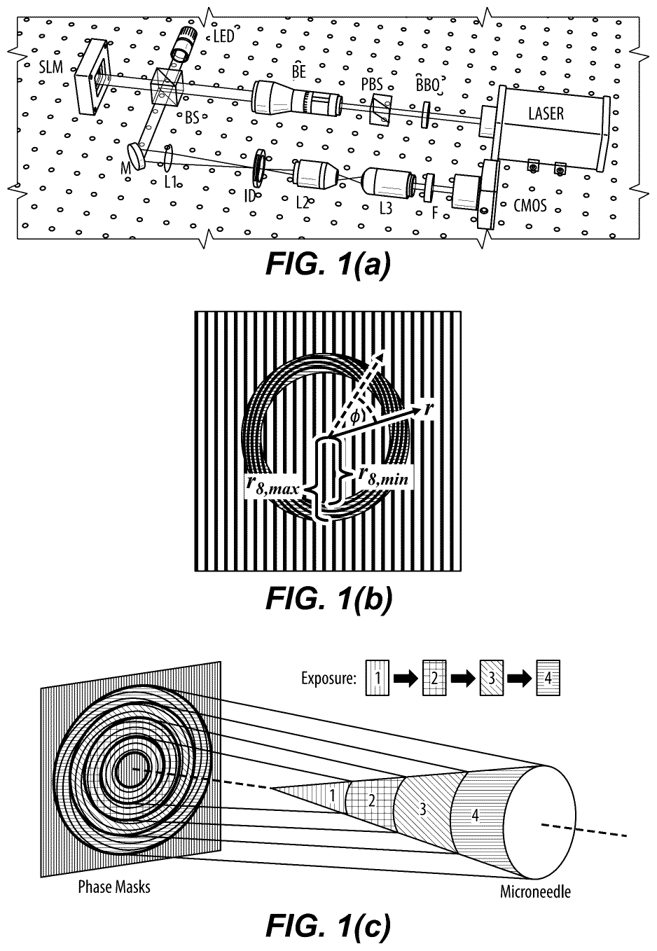

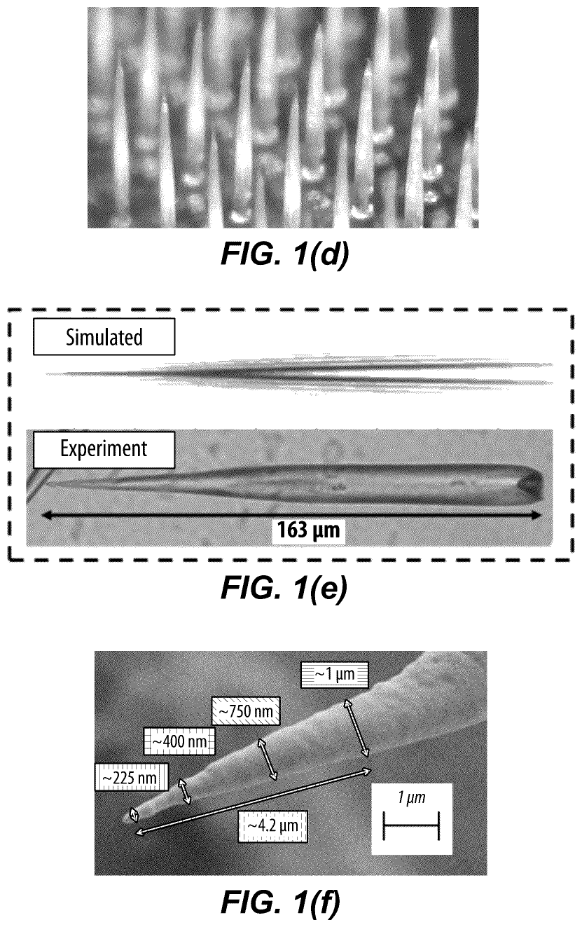

Reference will now be made to the drawings wherein like reference numerals identify similar structural features or aspects of the subject disclosure. For purposes of explanation and illustration, and not limitation, a partial view of an embodiment of a system in accordance with the disclosure is shown in FIG. 1(a) and is designated generally by reference character 100. Other embodiments of systems in accordance with the disclosure, or aspects thereof, are provided in FIG. 1(b)-8(d), as will be described. The systems and methods described herein can be used to form structures volumetrically, that are contoured relative to the optical axis of the beam forming the structures.

Micro-needles (MNs) are a type of transdermal drug delivery device that can be fabricated by serially-exposed multi-photon lithography (MPL). However, it is often time-consuming to fabricate large MN arrays using MPL because the sharpness of the MN tip requires high-resolution patterning, which in turn reduces fabrication speed. In this disclosure, we report a new microfabrication method based on three-dimensionally (3D) constructed light fields that can be used to fabricate MNs with a small number of exposures and therefore significantly reduced fabrication time. We describe the construction of the 3D light fields, special sample preparation/development procedures for volumetric MPL, examples of fabricated MNs and MN arrays, and unexpected results of this new microfabrication paradigm. Our method can be used to fabricate large MN arrays quickly and can be expanded to fabricate other microstructures for applications in medicine, photonics and other fields.

Micro-needles (MNs) and micro-needle arrays are commonly used in medicine and healthcare as an alternative means of drug delivery with no or reduced pain to the patient. MNs can be made of a variety of materials including metal, semiconductor, glass and polymer, using fabrication methods such as laser micromachining, photolithography, and chemical etching. Among these methods, multiphoton lithography (MPL) such as two-photon polymerization (TPP) or the like, is a direct laser writing method that uses pulsed lasers (often ultrafast lasers) to cause monomers to cross-link to form solid polymer structures at predetermined locations. TPP has been used to fabricate MNs. However, the main drawback of TPP is its slow fabrication speed, originating from the fact that the method is a serial exposure process in which only the focal point of the laser beam (usually with a volume on the order of 1 μm³) initiates the chemical reaction of photopolymerization. A 7×7 MN array can take hours to fabricate using traditional techniques, and while the fabrication time could be shortened by changing processing parameters, quality of the MNs is reduced when using traditional techniques.

To increase fabrication speed while maintaining good structure quality, we have reported recently a method that can fabricate microstructures “volumetrically” using three-dimensionally (3D) structured light field. In this method, a normal Gaussian ultrafast laser beam is incident on a spatial light modulator (SLM) that imposes phase change (“phase mask”) to the transverse profile of the beam and transforms the beam into a superposition of high-order Bessel modes. Phase masks designed with the theoretical framework developed in that work can produce “helical beams” with high intensity regions rotating as the beams propagate, resulting in an entire helical structure with tunable transverse and longitudinal shape formed “instantaneously” without scanning. With this method, we have fabricated large (30×30) helix arrays in 15 min. Further improvement in fabrication speed is possible with faster beam scanning and optimized laser exposure conditions.

This disclosure describes using a 3D beam shaping method for volumetric fabrication of MNs, which can be applied in medicine and other applications. This disclosure describes the optical design of the fabrication system, sample preparation, fabrication procedure, and finally measurement and assessment of the fabricated MNs. This work increases understanding of volumetric microfabrication using 3D structured light and demonstrates how MPL with 3D structured beams and constructed exposures can be used for scalable fabrication of microstructures for medical and other applications.

A phase mask (also called computer generation hologram or CGH) can be divided into multiple annular sections, and each section is “projected” at different axial (z) locations. The Bessel beam phases of various orders l for these sections are applied and combined with a phase corresponding to an axicon, as shown in FIG. 1(b).

The transmittance function of a specific annulus section on the SLM phase mask can be written as

$$t ( r , φ ) = {e}^{- ik ⊥ r} {e}^{il φ} , \qquad ( Eq . 1 )$$

where (r, φ) is the polar coordinates on the phase mask; k_{⊥} is the transverse component of the wave vector k_{⊥}=k sin α, where k=2π/λ is the wavenumber at vacuum wavelength λ, where α is angle of rays converging on the optical axis determined by an axicon base angle β and refractive index n, α=sin⁻¹(n sin β)−β, and l=0, 1, 2 . . . is the order of the Bessel mode. When projected, this phase mask will generate an intensity distribution in the focal region that resembles a shape cone, as shown in FIG. 1(c). Normally, a transmittance function in the form of Eq. 1 will generate a cylinder-like beam with parallel sidewalls. In our case, diffraction plays a significant role since each annulus section is a few mm wide, and the generated beam has a natural diverging angle which is beneficial for generating cone-like structures (discussed below).

The phase mask is displayed on an SLM as shown in FIG. 1(a). The other parts of the experimental setup are described below. The laser source is a femtosecond laser (LASER), e.g. Light Conversion, Pharos, that emits laser pulses of 170 fs pulse duration (full width at half maximum, FWHM), 1030 nm center wavelength, 6 W maximum average power, and 4 mm beam diameter (1/e² intensity level). The beam goes through a BBO crystal (BBO) for second harmonic generation of 515 nm light. A polarizing beamsplitter (PBS) removes the fundamental 1030 nm light, and a beam expander (BE) is used to expand the beam diameter to overfill the aperture of the SLM. A nonpolarizing beam splitter (BS) and a mirror (M) are used to direct the SLM-modulated beam to a telescope consisting of a lens (L1) and a microscope objective (L2) to reduce the size of the beam and increase its peak intensity. An iris diaphragm (ID) is used to block undesired high-order beams diffracted from the SLM. The phase mask shown in FIG. 1(b) is superposed with a grating phase in order to steer high-order beams so they can be blocked by ID. A sample is placed at the focal plane of the lens L2. The sample is a piece of glass slide spin coating with the photoresist (sample preparation procedure is explained later). A microscope consisting of another objective lens (L3), a long-pass filter to block the 515 nm beam, and a camera (CMOS) is used to monitor the beam shape and fabrication process. The microscope is illuminated with an infrared illuminator (LED) placed behind the BS.

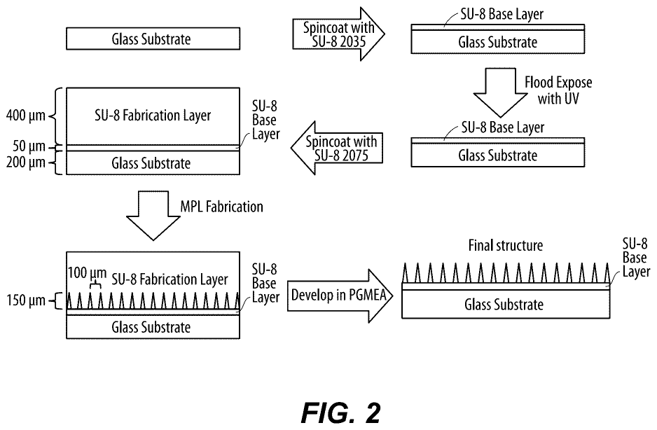

The sample preparation procedure is described below, and spin-coating recipes are described in Table 1. Procedures 1(a) and 1(b) both have an initial step at a lower spin speed followed by a second step at a higher spin speed and longer holding time. Procedure 1(c) only has one step, so spin speed, acceleration, and hold time for step 2 are not applicable. A flowchart outlining the sample preparation process is shown in FIG. 2. Microscope cover slips (25 mm×25 mm, thickness #2, Corning) were used as glass substrates. The substrates were washed with acetone and wiped dry with anti-static wipes. Omnicoat (Kayaku Advanced Materials) was then spin-coated onto the microscope cover slips to be used as an adhesion promoter to increase bonding strength between the substrate and the first layer of photoresin (see TABLE 1(a) for spin-coating recipe). Following spin-coating, substrates were placed on a hotplate, preheated to 200° C. Substrates were placed on the hotplate for 2 minutes. Following application of the adhesion promoter, SU-8 2035 (Kayaku Advanced Materials) was applied to each substrate and spin-coated (see Table 1(b)). This layer of photoresin was applied to fabricate a 50 μm-thick base layer of polymer, acting as a substrate for the needles. Samples were then soft-baked at 65° C. for 3 minutes, and 95° C. for 5 minutes. When soft-baking, samples were slowly brought up to the appropriate temperature for each step. Following soft-baking, the samples were placed under a long-pass filter and exposed to a UV floodlamp for 10 minutes. After flood exposure, the base layer of SU-8 was then post-exposure baked (PEB) at 65° C. for 3 minutes and 95° C. for 5 minutes.

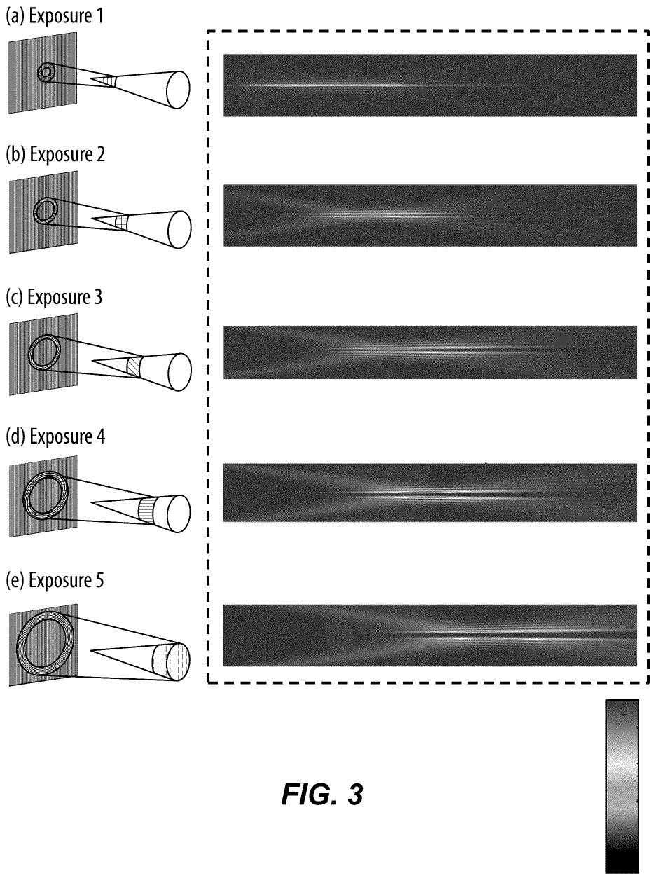

Following administration of the adhesion-promotion layer and polymer substrate layer, SU-8 2075 (Kayaku Advanced Materials) was then dispensed onto the samples to act as the fabrication layer of SU-8 in which the needles were fabricated. After ensuring samples were adequately covered by SU-8, samples were then spin-coated using the recipe outlined in Table 1(c). Upon removal from the spin-coater, samples were sprayed with a fine mist of acetone to remove edge bead and improve surface uniformity, following established methods. Samples were then placed on a hotplate, which was slowly brought up to 65° C., and covered. After leaving the samples for 1 hour, the temperature was slowly raised to 95° C. and samples were left to soft-bake for 1 hour. To ensure soft-baking was completed, samples were prodded with forceps to see if the surface of the resin was tacky or soft. If the soft-baking step was not completed, samples were returned to the hotplate at 95° C. and left to continue soft-baking until completed, being monitored every 10 minutes. Upon completion of soft-baking, samples were moved to the optical fabrication system outlined in FIG. 1(a). If a thicker fabrication layer was desired, the recipe shown in Table 1(c) was repeated prior to fabrication. FIG. 3 is a simulated x-z intensity map in the focal region for the system of FIG. 1(a), showing the five segments used to construct a cone-shaped beam. FIG. 1(d) is a photograph of an array of cones structures formed using the system of FIG. 1(a), FIG. 1(e) is a side-by-side comparison of an image of a simulated structure and a corresponding structure formed using the system of FIG. 1(a), and FIG. 1(f) is a photograph of a structure formed using the system of FIG. 1(a), showing a size scale for reference.

Samples were mounted to a motorized stage (MP-285A, Sutter Instruments) and leveled by moving the stage along both transverse axes and adjusting the mount to ensure the sample remained in-focus during stage travel. The CMOS camera was then moved to ensure the beam was in-focus while the phase mask corresponding to the base of the needle was displayed on the SLM. The stage was moved along the direction of beam propagation until the SU-8/glass interface was in-focus, which was done by imaging dust particles. The stage was then moved 50 μm to compensate for the thickness of the substrate layer, and the fabrication process was then performed. Needles were fabricated by sequentially displaying each phase mask on the SLM and sending laser pulses through the system. Following fabrication, a PEB step was performed on the samples by heating at 65° C. for 10 minutes, followed by heating at 95° C. for 1 hour. Samples were then developed using propylene glycol methyl ether acetate (PGMEA) as a developer. Samples were bathed in developer for 20 minutes, which was then slowly drained and replaced. The development process was performed for a total of 3×20-minute baths. The sample was then covered with isopropyl alcohol and drained slowly to remove residual PGMEA.

### Generation of Needle Beams

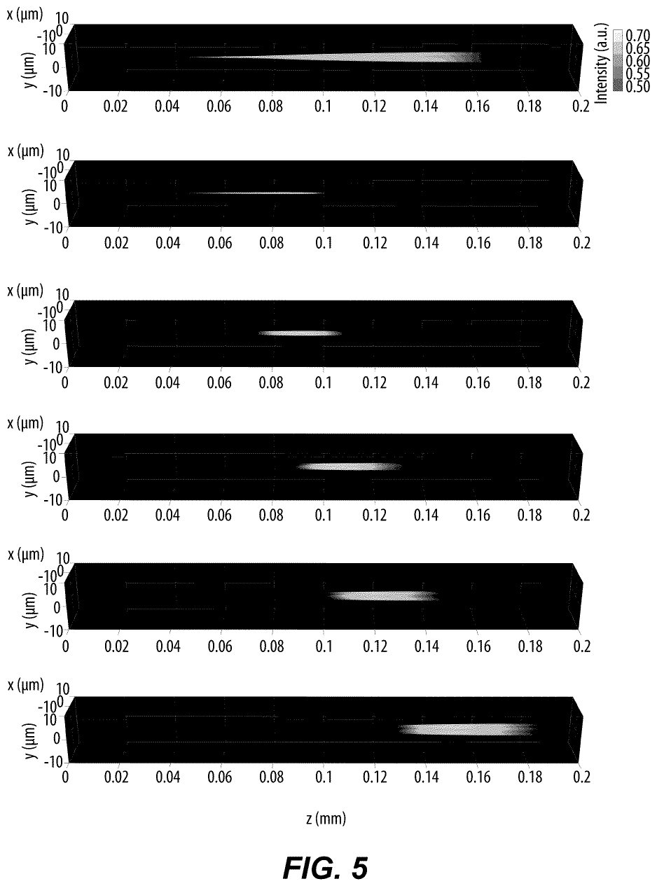

The key to the laser fabrication of MNs at high throughput is to eliminate beam scanning as much as possible. This requires a beam shaping procedure that can adaptively change the shape of the beam according to desired structural shape. In this study, we combine multiple “beam segments” with tunable shape to obtain a final beam shape suitable for MN fabrication. FIG. 5 shows simulated laser intensity in the x-z plane, of the combined beam (FIG. 5, top most) and the five constituting segments. In a typical configuration, the “tip” of the needle is made of the zero-order Bessel beam l=0, as shown in FIG. 5. This is to ensure that the MN has a sharp tip. The other segments are made of Bessel modes with higher order l>1. A standard high-order Bessel beam has intensity resembling a hollow tube. In our study, the shape has a divergent angle due to light diffraction, as shown in FIG. 5 (five constituents below the top-most) and will be discussed more in the next section. This is normally considered as a drawback, whereas in our study, the divergence makes it possible to “stitch” different segments to form continuous final shape. To further smoothen the final shape, the annulus segments are partially overlapped on the SLM. The amount of overlapping is 10-50% of the annulus width. FIG. 5 (top most) shows a cone-shaped beam combined from the five segments by adding the intensity directly. We can see that the leading (tip) part of the beam has higher intensity than the trailing part. This is due to the incident Gaussian beam with higher intensity in the central region. To improve the beam shape, we assign different numbers of pulses (N) to each segment, so the “total intensity” (Σ NI) is more uniform across different segments, as shown in FIG. 3(g). As will be discussed later, we also consider absorbed dose (D∝Σ NI²) as the factor to assess the quality of generated beam shapes.

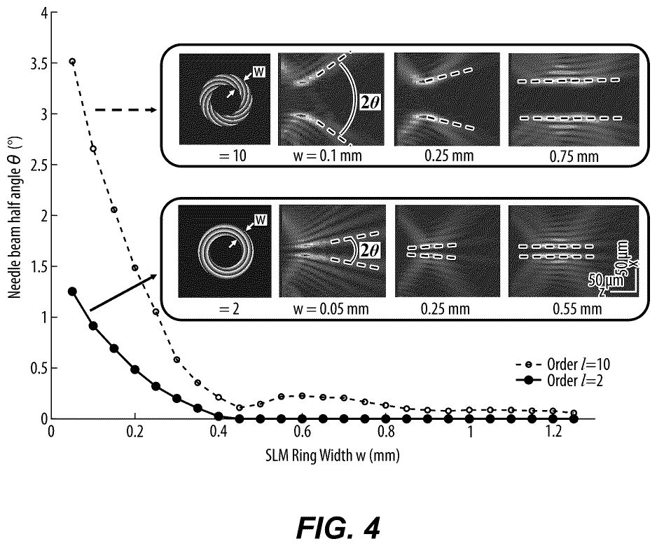

As discussed above, we utilize the inherent diffraction effect to generate the cone-like beam shape. This is achieved by varying the width of an annulus ring in the phase mask. This is illustrated in FIG. 4, which shows how the ring width (w) affects the half angle of the needle beam (θ) for two exemplary orders (l). θ was first determined using a computer algorithm that looks for the highest intensity at each z-position, and then finds θ by connecting these maximum intensity positions. Because the beam shape is not a perfect cone, this method does not always produce results that match the overall shape of the beam. Therefore, we chose to use a more subjective method in which we visually inspect the beam shape and draw contour lines that best match the overall shape of the beam. We have verified that two methods produce results that qualitatively match each other.

We can see from FIG. 4 that, first, a higher order (l=10) produces a beam that is wider along the x-axis. This is normal behavior of high-order Bessel modes. Second, the cone half angle θ increase with reducing the ring width w of the phase mask for both orders l. We have verified this trend for other orders, and attribute this to natural light diffraction. Third, the largest full angle that we can produce is approximately 2θ=7° at w=0.05 mm. Reducing w further decreases beam quality because of the definite size of the SLM pixels (9.2 μm).

To better visualize the beam shape, FIG. 5 (top most) shows the iso-intensity surface rendered in 3D. We can see that the combined beam has a sharp tip and gradually increases in diameter as z increases.

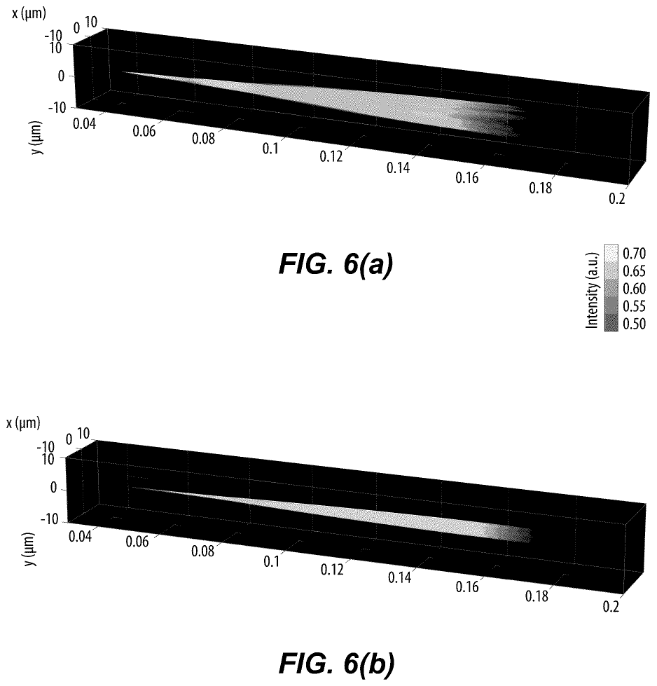

As discussed above, the shape of these beams can be tuned by changing the SLM phase mask. FIGS. 6(a) and (6b) show cones with different cone angles. It is found that cones with large cone angles have more distortion than cones with smaller angles. This is attributed to the finite numerical aperture of the optical system.

### Fabrication Result and Discussion

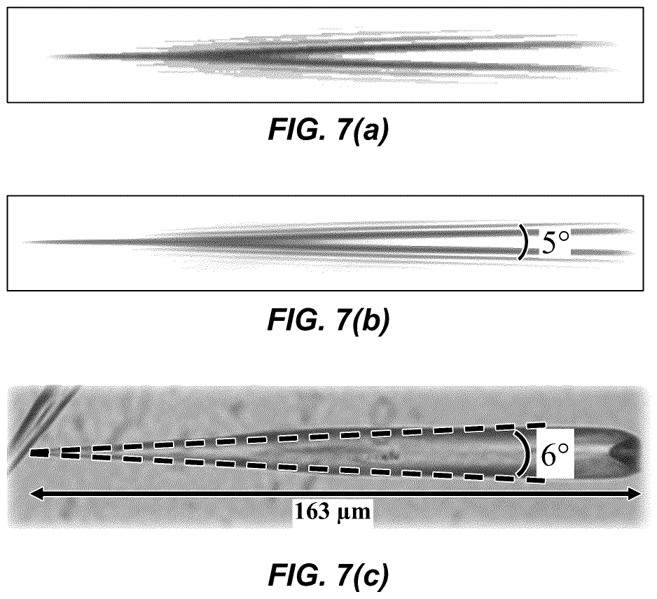

We first compare the shape of fabricated MNs with simulation. FIG. 7(a)-7(c) show a comparison of simulated beam shape (intensity I, FIG. 7(a)), absorbed dose (D∝NI², FIG. 7(b)), and a MN observed under optical microscope (FIG. 7(c)). The scales of these three images have been adjusted to be identical. This particular MN falls flat on the glass substrate due to poor adhesion, which inadvertently helps us to examine its shape easily. The simulated beam has a cone angle of 5°, and is targeted to fabricate a MN of 150 μm long. From FIG. 7(c), we can see that the fabricated MN has a cone angle of 6° and length of 163 μm. The difference could be due to shrinkage during development. The “base” part of the MN deviates from the design and has parallel sidewalls. Another unexpected feature is the internal of the MN which appears to be solid (will be confirmed below). Aside from the shrinkage issue mentioned above, other mechanisms possibly responsible for these anomalies are species diffusion, reflection from the interface(s), etc. Nevertheless, the fabricated MN has a dimension and shape similar to the design.

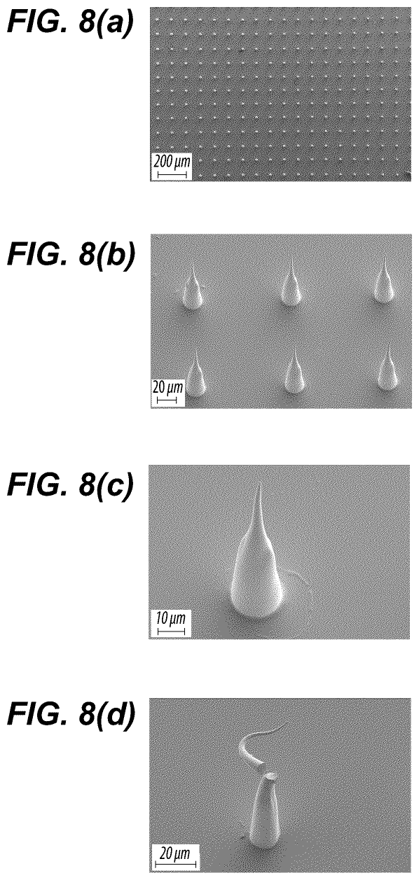

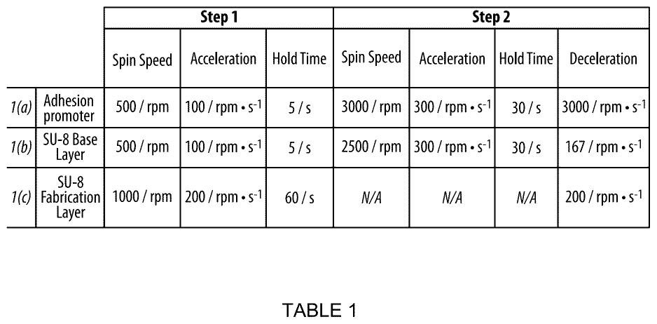

In another round of experiments, we attempt to fabricate MN arrays using a “step and exposure” method, i.e., sequentially fabricating each MN after moving the substrate to new location using a motorized stage. FIG. 8(a)-8(d) show SEM images of a MN array and some magnified views of individual MNs. We have successfully fabricated MN arrays with size as large as 10×5 mm, consisting of 100×50 MNs. Fabrication time is 15 hrs. A majority (70%) of the fabrication time is spent on translating the substrate on the motorized stage. It is possible to reduce the fabrication time by using scanning optics such as the galvanometer.

Zoomed-in images (FIG. 8(b-c)) of the MN array show that individual MNs have sharp tips and stand upright with a height of approximately 50 μm-300 μm. We have deliberately increased exposure dose of the base portion of the MNs to improve adhesion. As a consequence, the base portion is wider than that shown in FIG. 5. FIG. 8(d) shows a MN fabricated with a lower dose and broken unintentionally during development. We can see that the upper portion becomes hair-like due to reduced rigidity. We also notice from the broken interface that the MN has a solid internal, at least in the portion exposed in the break point.

In an effort to speed up the fabrication of large microneedle (MN) arrays, we have demonstrated in this paper a proof of concept of a new multiphoton lithography (MPL) method based on three-dimensionally (3D) constructed light fields called the “needle beam”. A needle beam is constructed from a set of exposures each of which forms a segment of a needle using a mapping relationship between the transverse plane of a phase mask and different axial locations of the target fabrication region. We have shown that the shape of such needle beams can be tuned within the limit of the optical system, and have used such beams to fabricate MN arrays. We expect that this new MPL paradigm is a step towards applying MPL in industrial settings, and can promote other volumetric fabricate methods that address the speed issue of today's MPL.

The methods and systems of the present disclosure, as described above and shown in the drawings, provide for forming structures volumetrically, that are contoured relative to the optical axis of the beam forming the structures. While the apparatus and methods of the subject disclosure have been shown and described with reference to preferred embodiments, those skilled in the art will readily appreciate that changes and/or modifications may be made thereto without departing from the scope of the subject disclosure.

What is claimed is:

1. A method comprising:
illuminating a medium with optical illumination,
wherein the medium is optically curable to form a cured structure of a portion of the medium that is illuminated by a concentration of the optical illumination,
wherein the optical illumination is incident on the medium along an optical axis, and
wherein illuminating includes forming illumination that is both axially and transversely structured to form a volumetric structure in the medium that is contoured relative to the optical axis.

2. The method as recited in claim 1, wherein the optical illumination originates from an optical source, wherein the volumetric structure diverges relative to the optical axis in a direction extending away from the illumination source.

3. The method as recited in claim 1, wherein the optical illumination originates from an optical source, wherein the volumetric structure converges relative to the optical axis in a direction extending toward the illumination source.

4. The method as recited in claim 1, wherein the optical illumination originates from an optical source, wherein the volumetric structure is convex or concave relative to the optical axis.

5. The method as recited in claim 1, wherein the volumetric structure is defined about a hollow void that extends along the optical axis.

6. The method as recited in claim 1, wherein the volumetric structure is a micro structure.

7. The method as recited in claim 6, wherein the microstructure is a microneedle.

8. The method as recited in claim 7, wherein the microneedle is formed of a plurality of volumetric structures formed one upon another.

9. The method as recited in claim 1, wherein the volumetric structure is a first volumetric structure, and further comprising:
illuminating the medium to form a plurality of volumetric structures wherein the plurality of volumetric structures includes a grid of microneedles, each formed by a single respective firing of an illuminator to illuminate a different portion of the medium, wherein forming each microneedle in the grid of microneedles includes positioning at least one of the medium or an optical element of a system forming the illumination for each respective firing of the illuminator.

10. The method as recited in claim 1, wherein illuminating the medium includes transmitting the illumination from an illumination source, through a modulating device, and from the modulating device into the medium, wherein the modulating device displays a phase mask to selectively block and transmit portions of the illumination from the illuminator to the medium, wherein the phase mask is predetermined to form the volumetric structure.

11. The method as recited in claim 10, wherein the phase mask includes a circumferentially distributed set of spiraling curve segments.

12. The method as recited in claim 10, wherein the volumetric structure is a first volumetric structure in a sequence of volumetric structures and further comprising:
repeating illumination for each of at least one volumetric structure in the sequence of volumetric structures to stack each of the at least one volumetric structure upon a previous one of the volumetric structures in the sequence of volumetric structures starting from the first volumetric structure to form a stacked structure.

13. The method as recited in claim 12, wherein each volumetric structure in the plurality of volumetric structures in the stacked structure has a respective predetermined contour relative to the optical axis based on a respective phase mask used for each repeat of illumination, wherein each respective predetermined contour is formed by a respective phase mask pattern on the modulating device.

14. The method as recited in claim 10, wherein a transmittance function for an annulus section of the phase mask is
$$t ( r , φ ) = {e}^{- ik ⊥ r} {e}^{il φ} ,$$
where (r, φ) is polar coordinates on the phase mask, k_{⊥} is a transverse component of the wave vector k_{⊥}=k sin α, where k=2π/λ is a wavenumber at vacuum wavelength λ, where α is angle of rays converging on the optical axis determined by an axicon base angle β and refractive index n, α=sin⁻¹(n sin β)−β, and l=0, 1, 2 . . . is an order of a Bessel mode, so that the phase mask will generate an intensity distribution in a focal region of the medium that is conical or frustoconical.

15. A system comprising:
an illuminator configured to emit a beam of illumination with a peak wavelength in a bandwidth;
a modulating device down an optical path from the illuminator for receiving the beam of illumination from the illuminator and configured to alter the beam of illumination into altered illumination;
a layer of fabrication medium down the optical path from the modulating device, wherein the optical path extends through the layer of fabrication medium along an optical axis; and
a controller configured to:
use the illuminator to illuminate a portion of the layer of fabrication medium with the beam of illumination altered by the modulating device, wherein the fabrication medium is optically curable within the bandwidth to form a cured structure of the portion of the layer of fabrication medium that is illuminated by the altered illumination, wherein the beam of optical illumination is incident on the layer of fabrication medium along an optical axis, and
use the modulating device to concentrate the beam of optical illumination in the layer of fabrication medium to form a volumetric structure in the layer of fabrication medium that is contoured relative to the illumination axis.

16. The system as recited in claim 15, further comprising a telescope that includes:
a lens down the optical path from the modulating device; and
a microscope objective down the optical path from the lens, wherein the fabrication medium is down the optical path from the microscope objective, and wherein the lens and microscope objective are configured to reduce size of the beam of illumination and increase peak intensity of the beam of illumination.

17. The system as recited in claim 16, further comprising:
a β phase barium borate (BBO) crystal down the optical path from the illuminator for second harmonic generation;
a polarizing beam splitter (PBS) down the optical path from the BBO crystal for removing fundamental light; and
a beam expander (BE) down the optical path from the PBS, wherein the modulating device is down the optical path from the BE for expanding the beam of illumination to a diameter to overfill an aperture of the modulating device.

18. The system as recited in claim 17, further comprising:
a beam splitter between the BE and the modulating device, configured to pass the beam of illumination from the BE through the beam splitter to the modulating device, and to reflect a return of the beam of illumination from the modulating device toward the lens;
a mirror down the optical path from the beam splitter configured to reflect the beam of illumination from the beam splitter to the lens; and
an iris diaphragm (ID) down the optical path from the lens for blocking higher-order beams diffracted from the modulating device, wherein the microscope objective is down the optical path from the ID.

19. The system as recited in claim 18, further comprising a monitor microscope that includes:
a monitoring microscope objective lens down the optical path from the layer of fabrication medium;
a filter down the optical path from the monitoring microscope objective lens for blocking illimitation in the bandwidth of the illuminator;
a camera down the optical path from the filter; and
a camera illuminator directing camera illumination into the optical path through the beam splitter, through the telescope, through the layer of fabrication medium, through the monitor microscope objective lens, and through the filter into the camera for imaging structures in the fabrication medium.

20. The system as recited in claim 15, wherein the controller is configured to control the phase mask to have a transmittance function for an annulus section of the phase mask given by
$$t ( r , φ ) = {e}^{- ik ⊥ r} {e}^{il φ} ,$$
where (r, φ) is polar coordinates on the phase mask, k_{⊥} is a transverse component of the wave vector k_{⊥}=k sin α, where k=2π/λ is a wavenumber at vacuum wavelength λ, where α is angle of rays converging on the optical axis determined by an axicon base angle β and refractive index n, α=sin⁻¹(n sin β)−β, and l=0, 1, 2 . . . is an order of a Bessel mode, so that the phase mask generates an intensity distribution in a focal region of the fabrication medium that is conical or frustoconical.
